(19) United States
(12) Patent Application Publication
(10) Pub. No.: US 2014/0350097 A1
(43) Pub. Date: Nov. 27, 2014

(54) TREATMENT OF HYPOTENSION ASSOCIATED WITH HEMODIALYSIS

(71) Applicant: Medinox,Inc., Carlsbad, CA (US)

(72) Inventor: Ching-San Lai, Carlsbad, CA (US)

(73) Assignee: Medinox,Inc., Carlsbad, CA (US)

(21) Appl. No.: 13/901,323

(22) Filed: May 23, 2013

(51) Int. Cl. A61K 31/27; G01N 33/50; A61K 31/165

(52) U.S. Cl. CPC A61K 31/27; A61K 31/165; G01N 33/50
USPC 514/476; 514/626; 436/116; 436/110

(57) ABSTRACT

There is provided a method for the prevention and/or treatment of hypotension associated with hemodialysis, which method includes administering to a subject in need thereof an effective amount of at least one physiologically compatible compound which, in combination with iron, binds nitric oxide. There is further provided a method for identifying hemodialysis subjects for whom treatment with an effective amount of at least one physiologically compatible compound which, in combination with iron, binds nitric oxide is indicated. The method includes monitoring the subject's nitric oxide levels, and selecting those subjects having elevated nitric oxide levels for treatment with an effective amount of at least one physiologically compatible compound which, in combination with iron, binds nitric oxide, wherein the effective amount is sufficient to maintain the subject's nitric oxide levels within an acceptable range.

### FIELD OF THE INVENTION

The present invention relates to methods for reducing hypotension associated with hemodialysis by administering to a subject in need thereof an effective amount of at least one physiologically compatible compound which, in combination with iron, binds nitric oxide. The present invention further relates to identification of subject subpopulations for whom a reduction in hypotension associated with hemodialysis can be achieved by administering to a subject in need thereof an effective amount of at least one physiologically compatible compound which, in combination with iron, binds nitric oxide.

### BACKGROUND OF THE INVENTION

Nitric oxide (.NO) is a free-radical cell messenger with numerous biological functions, including regulation of vascular tone, regulation of cellular signaling in the brain, and killing of pathogens by way of non-specific immune response (see, e.g., Ignarro, L. J., 1990, Ann. Rev. Toxicol 30:535-560; Moncada, S., 1992, Acta. Physiol. Scand. 145:201-227; and Lowenstein & Snyder, 1992, Cell 70:705-707). As known in the art, .NO is the product of the five-electron enzymatic oxidation of one of the chemically equivalent guanidino nitrogens of L-arginine. This oxidation is catalyzed by the enzyme, nitric oxide synthase. As known in the art, four major types of nitric oxide synthase have been identified: neuronal NOS (nNOS), inducible NOS (iNOS), endothelial NOS (eNOS), and bacterial NOS (bNOS).

Endothelial .NO synthase (eNOS) is present in the endothelium, where .NO, a potent vasodilator, is continuously generated at low concentrations to regulate blood pressure and vascular tone. Inducible .NO synthase is present in many cell types, including macrophages, neutrophils and leukocytes, as well as hepatocytes, vascular endothelial and smooth muscle cells. This .NO synthase is induced by lipopolysaccharide (LPS) and cytokines, and produces .NO at high concentrations for several days, serving important roles in non-specific immunity against inflammation and infection (see e.g., Kilbourn & Griffith, 1992, J. Natl. Cancer Instit., 84:827-831; Moncada & Higga, N., 1993, Eng. J. Med., 329:2002-2012). In the case of severe infection, overproduction of .NO and cytokines can lead to life-threatening hypotension, multiple organ failure and eventually death. See e.g., St. John & Dorinsky, 1993, Chest 103:932-943.

In blood, .NO produced by the endothelium diffuses isotropically through all directions into adjacent tissues. As .NO diffuses into the vascular smooth muscle, it binds to guanylate cyclase enzyme, which catalyzes the production of cGMP, and induces vasodilation (see, e.g., Ignarro, L. J., Id.; Moncada, S., Id.; and Lowenstein & Snyder, Id.) Moreover, as .NO diffuses into the blood circulation, it reacts with hemoglobin in red blood cells to yield nitrate and methemoglobin (see e.g., Kelm & Schrader, 1990, Circ. Res. 66:1561-1575). Nitrate is eliminated via renal excretion and methemoglobin is enzymatically converted back into hemoglobin by methemoglobin reductase in red blood cells. It is therefore not surprising that serum nitrate levels are increased in cytokine-induced septic shock in animals and humans (see, e.g, Nava et al., 1992, J. Cardiovasc. Pharmacol. 20 (Suppl. 12):S132-134; Hibbs et al., 1992, J. Clin. Invest. 89:867-877; and Evans et al., 1993, Circulatory Shock 41:77-81).

Nitric oxide overproduction has been shown to cause systemic hypotension induced by LPS and cytokines (e.g., interleukin-1 (IL-1), interleukin-2 (IL-2), tumor necrosis factor (TNF) and interferons) (Kilbourn & Griffith, Id., Moncada & Higgs, Id.) Agents which inhibit the production of .NO (by inhibiting the action of nitric oxide synthase) have been studied as a means to treat systemic hypotension due to .NO overproduction. For example, N^{G}-monomethyl-L-arginine (NMMA), a competitive inhibitor of the nitric oxide biosynthetic pathway, was observed (upon intravenous injection) to reverse LPS-induced hypotension in animals (see e.g., Aisaka et al., 1989, Biochem. Biophys. Res. Commun., 60:881-886; Rees, et al., 1989, Proc. Natl. Acad. Sci. USA, 86:3375-3379). However, as noted in many recent reports, the inhibition of .NO synthase enzyme can be detrimental to the subject. See, e.g., Henderson et al., 1994, Arch. Surg. 129:1271-1275; Hambrecht et al., 1993, J. Leuk. Biol. 52:390-394; Luss et al., 1993, Biochem. and Biophys. Res. Comm. 204:635-640; Robertson et al., 1994, Arch. Surg. 129:149-156; Statman et al., 1994, J. Surg. Res. 57:93-98; Minnard et al., 1994, Arch. Surg. 129:142-148.

Thus, .NO plays important homeostatic roles in maintaining blood pressure, killing invading pathogens and cancer cells. Indeed, as known in the art, .NO additionally is implicated in facilitation of learning and memory. However, excessive .NO production has also been associated with numerous diseases and medical conditions including intradialytic hypotension (IDH), stroke, hemorrhagic shock, allograft rejection, diabetes, septic shock and cancer therapy with interleukin-2 (IL-2). Compounds disclosed herein have been shown both in vitro and in vivo to bind nitric oxide and neutralize its activities.

Intradialytic hypotension (IDH) is the most common adverse event of routine hemodialysis and in some instances is associated with the formation of excessive nitric oxide (.NO). A study reported that hemodialysis activates eNOS in erythrocytes to excessively produce .NO resulting in systemic hypotension. See Fischer U M et al., 2007, Ann., Thorac. Surg., 84:2000-2003). Thus, .NO-specific scavengers could alleviate IDH. At present, however, there are no effective drug therapies to prevent IDH, which occurs in up to 48% of all hemodialysis treatments. See e.g., Rosa et al, Arch. Intern. Med., 1980, 140:804-807; CLINICAL DIALYSIS, (3^{rd} Ed., Appleton & Lane) 1995:235-263. Current therapy for IDH involves, e.g., administration of bolus injections of saline, placing the patient in a reclining (Trendelenburg) position, administration of pressor agents such as dopamine, midodrine, and the like. None of these therapies, however, alleviates or prevents IDH by addressing the underlying mechanisms resulting in hypotension.

Indeed, published scientific studies have strongly linked excessive .NO production with IDH. These studies reported that .NO, a known potent vasodilator, is frequently generated during hemodialysis, as evidenced by high levels of blood nitrate, the end product of .NO metabolism. See e.g., Yokokawa K., et al, 1995, Ann. Intern. Med. 123:35-37; Lin, S. H., et al, 1996, ASAIO J 42:M895-M899; Kang, E., et al, 1997, Amer. J. Med. Sci. 313:138-146; Martensson, L., t al, 1997, Artif. Organs 21:163-167. Moreover, excessive .NO production has been associated with the dialysis procedure, resulting e.g., from activation of complement (see e.g., Chenoweth D. E., et al, 1983, Kidney Int. 24:764-769) and macrophages by the dialysis membranes (see e.g., Dodd, N., et al, 1982, Intern J. Artif Organ, 5:272-276; Amore, A., et al, 1995, J. Am. Soc. Nephrol. 6:1278-1283), heparin (see e.g., Yokokawa, K., et al, 1993, J. Clin. Invest. 92:2080-2085), endotoxin in the dialysate (see e.g., Lonnemann, G., 1988, Kidney Int. 33:29-35), or removal by dialysis of an endogenous inhibitor of nitric oxide synthase (NOS), the enzyme that orchestrates synthesis of .NO (see e.g., Kang 1997, Id.)

### BRIEF DESCRIPTION OF THE INVENTION

In accordance with the present invention, there is provided a method for the prevention and/or treatment of hypotension associated with hemodialysis. The method includes administering to a subject in need thereof an effective amount of at least one physiologically compatible compound which, in combination with iron, binds nitric oxide.

In another aspect, there is provided a method for the treatment of a subject in need thereof to prevent and/or minimize the occurrence of hypotension when the subject is undergoing hemodialysis, wherein the subject has nitrite/nitrate levels greater than 50 micromolar. The method includes administering to the subject an effective amount of at least one physiologically compatible compound which, in combination with iron, binds nitric oxide.

In another aspect, there is provided a method for identifying hemodialysis subjects for whom treatment is indicated with at least one physiologically compatible compound which, in combination with iron, binds nitric oxide. The method includes monitoring the subject's nitric oxide levels, and selecting those subjects having elevated nitric oxide levels for treatment. Treatment includes administration of an effective amount of at least one physiologically compatible compound which, in combination with iron, binds nitric oxide to maintain the subject's nitric oxide levels within an acceptable range.

In another aspect, there is provided a method for the prevention and/or treatment of hypotension by the in vivo reduction of nitric oxide levels in a subject undergoing hemodialysis and expressing elevated levels of endothelial nitric oxide synthase (eNOS). The method includes administering to the subject an effective amount of at least one physiologically compatible compound which, in combination with iron, binds nitric oxide.

In another aspect, there is provided a method for the prevention and/or treatment of nitric oxide overproduction in a subject undergoing hemodialysis and expressing elevated levels of endothelial nitric oxide synthase (eNOS). The method includes administering to the subject an effective amount of at least one physiologically compatible compound which, in combination with iron, binds nitric oxide.

In another aspect, there is provided a method for the prevention and/or treatment of hypotension in a subject undergoing hemodialysis and expressing elevated levels of endothelial nitric oxide synthase (eNOS). The method includes monitoring the subject's nitric oxide levels, and administering to those subjects having elevated nitric oxide levels a sufficient amount of at least one physiologically compatible compound which, in combination with iron, binds nitric oxide to maintain the subject's nitric oxide levels within an acceptable range.

In another aspect, there is provided a method to determine what treatment is indicated for a subject at risk of developing hypotension associated with hemodialysis. The method includes monitoring the subject's nitric oxide levels, and identifying those subjects which display an elevated level of nitric oxide as suitable for treatment with an effective amount of at least one physiologically compatible compound which, in combination with iron, binds nitric oxide.

In another aspect, there is provided a method to determine what treatment is indicated for a subject at risk of developing hypotension associated with hemodialysis. The method includes monitoring the subject's endothelial nitric oxide synthase levels, and identifying those subjects which display an elevated level of endothelial nitric oxide synthase as suitable for treatment with an effective amount of at least one physiologically compatible compound which, in combination with iron, binds nitric oxide.

In another aspect, there is provided a method to determine whether a subject at risk of developing hypotension associated with hemodialysis is receiving adequate treatment therefor. The method includes administering to those subjects having elevated nitric oxide an effective amount of at least one physiologically compatible compound which, in combination with iron, binds nitric oxide so as to bring the subject's nitric oxide levels within an acceptable range. The method further includes monitoring the subject's nitric oxide levels, and adjusting the dosage of the physiologically compatible compound which, in combination with iron, binds nitric oxide as needed.

In another aspect, there is provided a unit dosage form including a sufficient amount of a physiologically compatible compound which, in combination with iron, binds nitric oxide for administration thereof to a subject in need thereof having hypotension associated with hemodialysis, and a pharmaceutically acceptable carrier therefor.

In another aspect, there is provided a composition including a physiologically compatible compound which, in combination with iron, binds nitric oxide, and midodrine.

In another aspect, there is provided a method for the prevention and/or treatment of hypotension associated with hemodialysis. The method includes administering to a subject in need thereof an effective amount of a composition including a physiologically compatible compound which, in combination with iron, binds nitric oxide, and midodrine.

In another aspect, there is provided a method for the prevention and/or treatment of hypotension associated with hemodialysis. The method includes administering to a subject in need thereof an effective amount of at least one physiologically compatible compound which, in combination with iron, binds nitric oxide, and midodrine, wherein the compound and midodrine are administered separately or together.

In another aspect, there is provided a kit including a physiologically compatible compound which, in combination with iron, binds nitric oxide, a pharmaceutically acceptable carrier therefor, and instructions for the use thereof in the treatment of a subject in need thereof having hypotension associated with hemodialysis.

### BRIEF DESCRIPTION OF THE DRAWINGS

FIG. 1 depicts the results of plasma Cₘₐₓ of NOX-100 after bolus administration in hemodialysis subjects as a function of dose. See Example 4.

FIG. 2 depicts the results of Area Under the Curve (AUC) of NOX-100 after bolus administration in hemodialysis subjects as a function of dose. See Example 4.

FIG. 3 depicts a histogram of plasma clearance of NOX-100 during dialysis after bolus administration in hemodialysis subject as a function of dose. Legend (x-axis, left to right): 0.33, 1, 3, 9, 25 and 50, mg/kg NOX-100. See Example 4.

### DETAILED DESCRIPTION

In accordance with the present invention, there is provided a method for the prevention and/or treatment of hypotension associated with hemodialysis. The method includes administering to a subject in need thereof an effective amount of at least one physiologically compatible compound which, in combination with iron, binds nitric oxide. Disease states contemplated herein include hypotension associated with hemodialysis; the terms “hypotension associated with hemodialysis,” “hemodialysis-related hypotension,” “intradialytic hypotension (IDH)” and the like refer to clinically observed hypotension in a population of subjects undergoing hemodialysis. It is understood that not all subjects undergoing hemodialysis present with hypotension associated with hemodialysis. Thus, it is understood that hypotension associated with hemodialysis presents in a subpopulation of subjects undergoing hemodialysis.

The methods of the present invention are directed to treating and/or preventing disease states in a subject caused or exacerbated by the presence of excess nitric oxide (.NO) in the subject. The methods include administering a pharmaceutically effective amount of at least one physiologically compatible compound which, in combination with iron, binds nitric oxide. It is understood that the term “binds nitric oxide” in this context refers to physiological sequestration of nitric oxide by which physiological nitric oxide activity is reduced. The terms “effective amount,” “pharmaceutically effective amount,” “sufficient amount” and the like refer to an amount sufficient to achieve its intended purpose, e.g., reduction in physiological levels of .NO, or amelioration or prevention of hypotension.

The terms “treatment,” “treating” and the like refer to any indicia of success in the prevention, treatment or amelioration of an injury, pathology or condition, including any objective or subjective parameter such as abatement, remission, diminishing of symptoms or making the injury, pathology or condition more tolerable to the patient, slowing in the rate of degeneration or decline, making the final point of degeneration less debilitating, improving a subject physical or mental well-being, and the like. The prevention, treatment or amelioration of symptoms can be based on objective or subjective parameters; including the results of a physical examination, laboratory tests, and the like. For example, the methods described herein may be used to treat the symptoms of hypotension, or to prevent the symptoms of hypotension.

Without wishing to be bound by any theory, it is believed that polymorphism in the endothelial nitric oxide synthase (eNOS) gene may contribute, at least in part, to differences in observed clinical levels of .NO expression by subjects undergoing hemodialysis, and that elevated levels of .NO result in hemodialysis-related hypotension. Irrespective of the underlying genetic basis, however, it is nonetheless observed that a certain first subpopulation of subjects undergoing hemodialysis develops clinical hemodialysis-related hypotension, and a certain second subpopulation thereof is observed to have elevated .NO levels in combination with hemodialysis-related hypotension. Accordingly, the present invention contemplates identification of this second subpopulation and prevention and/or amelioration of incident hemodialysis-related hypotension observed during hemodialysis in the absence of prophylaxis and/or treatment.

The terms “subject in need thereof” and the like refer to a subject presenting clinical parameters (e.g., having blood levels of specific physiological species before or during hemodialysis) which indicate that reduction in excess physiological levels of .NO would be beneficial in preventing, treating or otherwise ameliorating hypotension associated with hemodialysis. As known in the art, such clinical parameters include elevated levels of eNOS synthase, elevated levels of .NO, and/or elevated nitrite/nitrate levels. Clinical tests for determination of eNOS, .NO, and nitrite/nitrate levels are known in the art.

The terms “at least one physiologically compatible compound which, in combination with iron, binds nitric oxide” and the like refer to agents, e.g., physiologically compatible compounds disclosed herein, which have the capability of binding iron to provide an iron-bound chemical species which in turn binds .NO. The binding of .NO to the iron-bound chemical species can be reversible or, preferably, irreversible. The term “irreversible” in this context means that .NO is effectively sequestered physiologically by virtue of a dissociation rate of .NO which is slow relative to physiological clearance of the iron-bound chemical species binding .NO.

Without wishing to be bound by any theory, it is believed that a metal ion (e.g., iron, managanese cobalt, nickel, copper, zinc, and like) bound to a physiologically compatible compound disclosed herein can in turn bind .NO by mechanisms of binding known in the art, including, e.g., inner-sphere/outer-sphere metallic coordination. It is understood that the physiologically relevant combination of a physiologically compatible compound disclosed herein with a metal provides the chemical species which in turn binds .NO, effectively sequestering the .NO from physiological activity, e.g., clinical hypotension. Thus, the terms “nitric oxide scavenger” and the like refer to physiologically compatible compounds disclosed herein that can combine with a cation (e.g., iron or other metal) to yield a species which binds .NO.

In one embodiment, the method contemplates prevention of hypotension associated with hemodialysis. Thus, hypotension associated with hemodialysis is prevented in a subject in need thereof by administering to the subject an effective amount of at least one physiologically compatible compound which, in combination with iron, binds nitric oxide, optionally in combination with one or more agents known in the art of treatment or prevention of hypotension, e.g., midodrine.

Midodrine is a vasopressor/antihypotensive prodrug with structure following:

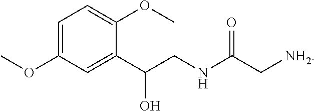 [SMILES: COc1ccc(OC)c(C(O)CNC(=O)CN)c1]

Upon physiological scission of the amide bond, midodrine affords desglymidodrine, which is understood to be the active agent with structure following:

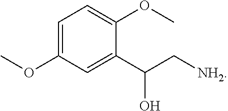

The active agent is an orally active α₁-receptor agonist which is believed to exert action via activation of the alpha-adrenergic receptors of the arteriolar and venous vasculature, producing an increase in vascular tone and elevation of blood pressure.

In one embodiment, the method contemplates treatment of hypotension associated with hemodialysis. Thus, hypotension associated with hemodialysis is treated in a subject in need thereof by administering to the subject an effective amount of at least one physiologically compatible compound which, in combination with iron, binds nitric oxide, optionally in combination with one or more agents known in the art of treatment or prevention of hypotension, e.g., midodrine.

In one embodiment, the at least one physiologically compatible compound which, in combination with iron, binds nitric oxide is administered to the subject prior to the manifestation of symptoms of hypotension.

In one embodiment, the subject in need thereof has elevated .NO levels. In one embodiment, elevated .NO levels are determined by measuring the subject's nitrite/nitrate levels. In one embodiment, elevated .NO levels are determined by measuring the subject's eNOS levels. In one embodiment, elevated .NO levels are determined by measuring the subject's .NO levels.

In one embodiment, elevated .NO levels are determined by measuring the subject's nitrite/nitrate levels, wherein the subject in need thereof has nitrite/nitrate levels greater than a specifically defined level. In one embodiment, the specifically defined level is in the range 30-100, 40-100, 50-100, 50-90, 50-80, 50-70 or 50-60 micromolar. In one embodiment, the specifically defined concentration is 30, 35, 40, 45, 50, 55, 60, 65, 70, 75, 80, 85, 90, 95, 100 micromolar, or even greater. In one embodiment, the subject in need thereof has nitrite/nitrate levels greater than 50 micromolar.

In one embodiment, the physiologically compatible compound which, in combination with iron, binds nitric oxide is a nitric oxide scavenger.

In one embodiment, the nitric oxide scavenger is a dithiocarbamate-containing nitric oxide scavenger. The terms “dithiocarbamate-containing nitric oxide scavenger” and the like refer, in the customary sense, to compounds containing at least one dithiocarbamate species, as well known in the art.

In one embodiment, the dithiocarbamate-containing nitric oxide scavenger has the structure of Formula (I) or (II) as follows:

[R¹R²N—C(S)—S⁻]ₓM^{+1, +2, +3} (I)

M^{+1, +2, +3}[⁻S—C(S)—NR¹]ₓ—R³—[R¹N—C(S)—S⁻]ₓM^{+1, +2, +3} (II).

With reference to Formula (I), each of R¹ and R² is independently selected from a C₁ up to C₁₈ alkyl, substituted alkyl, cycloalkyl, substituted cycloalkyl, heterocyclic, substituted heterocyclic, alkenyl, substituted alkenyl, alkynyl, substituted alkynyl, aryl, substituted aryl, heteroaryl, substituted heteroaryl, alkylaryl, substituted alkylaryl, arylalkyl, substituted arylalkyl, or R¹ and R² can cooperate to form a 5-, 6- or 7-membered ring including N, R¹ and R²; x is 1, 2 or 3, and M is a monovalent cation (e.g., H⁺, NH₄⁺, Na⁺ or K⁺, or the like) when x is 1, or M is a physiologically compatible divalent or trivalent transition metal cation (e.g., Co⁺², Co⁺³, Cu⁺², Fe⁺², Fe⁺³, Mn⁺², or Mn⁺³, or the like) when x is 2 or 3.

With reference to Formula (II), each R¹ is independently selected from a C₁ up to C₁₈ alkyl, substituted alkyl, cycloalkyl, substituted cycloalkyl, heterocyclic, substituted heterocyclic, alkenyl, substituted alkenyl, alkynyl, substituted alkynyl, aryl, substituted aryl, heteroaryl, substituted heteroaryl, alkylaryl, substituted alkylaryl, arylalkyl, or substituted arylalkyl; R³ is a divalent moiety selected from alkylene, substituted alkylene, oxyalkylene, substituted oxyalkylene, alkenylene, substituted alkenylene, arylene, substituted arylene, alkylarylene, substituted alkylarylene, aralkylene, or substituted aralkylene; x is 1, 2 or 3; and M is a monovalent cation when x is 1, or M is a physiologically compatible divalent or trivalent transition metal cation when x is 2 or 3.

In one embodiment, the dithiocarbamate-containing nitric oxide scavenger is combined with a transition metal and has the structure of Formula (Ia) or (IIa) as follows:

[R¹R²N—C(S)—S⁻]₂TM⁺² (Ia)

[⁻S—C(S)—NR¹]—R³—[R¹N—C(S)—S⁻]TM⁺² (IIa).

With reference to Formulae (Ia), R¹ and R² are as defined above for Formula (I). With reference to Formulae (IIa), R¹ and R³ are as defined above for Formula (II). Substituent TM is a transition metal, preferably iron.

The term “alkyl,” by itself or as part of another substituent, means, unless otherwise stated, a straight (i.e., unbranched) or branched chain hydrocarbyl, or combination thereof. An alkyl may designate the number of carbon atoms contemplated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like.

As employed herein, “substituted alkyl” comprises alkyl groups further bearing one or more substituents selected from hydroxy, alkoxy (of a lower alkyl group), mercapto (of a lower alkyl group), cycloalkyl, substituted cycloalkyl, heterocyclic, substituted heterocyclic, aryl, substituted aryl, heteroaryl, substituted heteroaryl, aryloxy, substituted aryloxy, halogen, trifluoromethyl, cyano, nitro, nitrone, amino, amido, —C(O)H, acyl, oxyacyl, carboxyl, carbamate, sulfonyl, sulfonamide, sulfuryl, and the like.

As employed herein, “cycloalkyl” refers to cyclic ring-containing groups containing in the range of about 3 up to 8 carbon atoms, and “substituted cycloalkyl” refers to cycloalkyl groups further bearing one or more substituents as set forth above.

As employed herein, “alkenyl” refers to straight or branched chain hydrocarbyl groups having at least one carbon-carbon double bond, and having in the range of about 2 up to 12 carbon atoms, and “substituted alkenyl” refers to alkenyl groups further bearing one or more substituents as set forth above.

As employed herein, “alkynyl” refers to straight or branched chain hydrocarbyl groups having at least one carbon-carbon triple bond, and having in the range of about 2 up to 12 carbon atoms, and “substituted alkynyl” refers to alkynyl groups further bearing one or more substituents as set forth above.

As employed herein, “aryl” refers to aromatic groups having in the range of 6 up to 14 carbon atoms and “substituted aryl” refers to aryl groups further bearing one or more substituents as set forth above.

As employed herein, “alkylaryl” refers to alkyl-substituted aryl groups and “substituted alkylaryl” refers to alkylaryl groups further bearing one or more substituents as set forth above.

As employed herein, “arylalky” refers to aryl-substituted alkyl groups and “substituted arylalky” refers to arylalkyl groups further bearing one or more substituents as set forth above.

As employed herein, “arylalkenyl” refers to aryl-substituted alkenyl groups and “substituted arylalkenyl” refers to arylalkenyl groups further bearing one or more substituents as set forth above.

As employed herein, “arylalkynyl” refers to aryl-substituted alkynyl groups and “substituted arylalkynyl” refers to arylalkynyl groups further bearing one or more substituents as set forth above.

As employed herein, “aroyl” refers to arylcarbonyl species such as benzoyl and “substituted aroyl” refers to aroyl groups further bearing one or more substituents as set forth above.

As employed herein, “heterocyclic” refers to cyclic (i.e., ring-containing) groups containing one or more heteroatoms (e.g., N, O, S, or the like) as part of the ring structure, and having in the range of 3 up to 14 carbon atoms and “substituted heterocyclic” refers to heterocyclic groups further bearing one or more substituents as set forth above.

As employed herein, “acyl” refers to alkylcarbonyl species.

As employed herein, “halogen” refers to fluoride, chloride, bromide or iodide atoms.

In one embodiment, the dithiocarbamate-containing nitric oxide scavenger has the structure of Formula (I); each of R¹ and R² is independently selected from a C₁ up to C₆ alkyl or substituted alkyl; x is 1; and M is a monovalent cation selected from NH₄⁺, Na⁺ or K⁺.

In one embodiment, the dithiocarbamate-containing nitric oxide scavenger has the structure of Formula (I); each of R¹ and R² is independently selected from a C₁ up to C₆ alkyl or substituted alkyl; x is 2 or 3; and M is selected from Fe⁺² or Fe⁺³.

In one embodiment, the dithiocarbamate-containing nitric oxide scavenger is N-methyl-D-glucamine dithiocarbamate (NOX-100), having structure following (as the sodium salt):

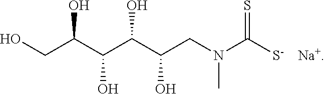 [SMILES: CN(C[C@H](O)[C@@H](O)[C@H](O)[C@H](O)CO)C(=S)[S-]]

In one embodiment, the at least one physiologically compatible compound which, in combination with iron, binds nitric oxide is administered to the subject prior to the manifestation of symptoms of hypotension.

In another aspect, there is provided a method for the treatment of a subject in need thereof to prevent and/or minimize the occurrence of hypotension when the subject is undergoing hemodialysis, wherein the subject has nitrite/nitrate levels greater than 50 micromolar. The method includes administering to the subject an effective amount of at least one physiologically compatible compound which, in combination with iron, binds nitric oxide, optionally in combination with one or more agents known in the art of treatment or prevention of hypotension, e.g., midodrine. Exemplary physiologically compatible compounds useful in the practice of this aspect of the invention include compounds with the structure of either of Formulae (I) or (II). Further exemplary physiologically compatible compounds useful in the practice of this aspect of the invention include compounds with the structure of either of Formulae (Ia) or (IIa). In one embodiment, the method contemplates preventing the occurrence of hypotension when the subject is undergoing hemodialysis. In one embodiment, the method contemplates minimizing the occurrence of hypotension when the subject is undergoing hemodialysis.

In one embodiment, the at least one physiologically compatible compound which, in combination with iron, binds nitric oxide is administered to the subject prior to the manifestation of symptoms of hypotension.

In another aspect, there is provided a method for identifying hemodialysis subjects for whom treatment with an effective amount of at least one physiologically compatible compound which, in combination with iron, binds nitric oxide is indicated. The method includes monitoring the subject's nitric oxide levels, and selecting those subjects having elevated nitric oxide levels for treatment. Treatment includes administration of a sufficient amount of at least one physiologically compatible compound which, in combination with iron, binds nitric oxide to maintain the subject's nitric oxide levels within an acceptable range. Methods for monitoring the subject's nitric oxide levels are well known in the art. The terms “elevated nitric oxide levels” and the like refer, in the customary sense, to levels of nitric oxide in the subject which are associated with hypotension. The terms “acceptable range” and the like in the context of nitric oxide levels of a subject refer, in the customary sense, to levels of nitric oxide which do not result in hypotension, or which result in blood pressure which is intermediate between normotensive and hypotensive. In one embodiment, the elevated nitric oxide levels are determined by measuring the subject's nitrite/nitrate levels.

In one embodiment, the physiologically compatible compound has the structure of any one of Formulae (I), (I), (Ia), or (IIa).

In another aspect, there is provided a method for the prevention and/or treatment of hypotension by the in vivo reduction of nitric oxide levels in a subject undergoing hemodialysis, wherein the subject is expressing elevated levels of endothelial nitric oxide synthase (eNOS). The method includes administering to the subject an effective amount of at least one physiologically compatible compound which, in combination with iron, binds nitric oxide, optionally in combination with one or more agents known in the art of treatment or prevention of hypotension, e.g., midodrine. In one embodiment, the method contemplates prevention of hypotension. In one embodiment, the method contemplates treatment of hypotension. The terms “elevated levels of endothelial nitric oxide synthase” and the like refer, in the customary sense, to levels of endothelial nitric oxide synthase (eNOS) which give rise to hypotension by increased production of .NO relative to the normotensive state.

In one embodiment, the at least one physiologically compatible compound which, in combination with iron, binds nitric oxide is administered to the subject prior to the manifestation of symptoms of hypotension.

In one embodiment, the physiologically compatible compound has the structure of any one of Formulae (I), (I), (Ia), or (IIa).

In another aspect, there is provided a method for the prevention and/or treatment of nitric oxide overproduction in a subject undergoing hemodialysis, wherein the subject is expressing elevated levels of endothelial nitric oxide synthase (eNOS). The method includes administering to the subject an effective amount of at least one physiologically compatible compound which, in combination with iron, binds nitric oxide, optionally in combination with one or more agents known in the art of treatment or prevention of hypotension, e.g., midodrine.

In one embodiment, the at least one physiologically compatible compound which, in combination with iron, binds nitric oxide is administered to the subject prior to the manifestation of symptoms of hypotension.

In one embodiment, the physiologically compatible compound has the structure of any one of Formulae (I), (I), (Ia), or (IIa).

In another aspect, there is provided a method for the prevention and/or treatment of hypotension in a subject undergoing hemodialysis, wherein the subject is expressing elevated levels of endothelial nitric oxide synthase (eNOS). The method includes monitoring the subject's nitric oxide levels, and administering to those subjects having elevated nitric oxide levels a sufficient amount of at least one physiologically compatible compound which, in combination with iron, binds nitric oxide to maintain the subject's nitric oxide levels within an acceptable range. Optionally, the physiologically compatible compound is administered in combination with one or more agents known in the art of treatment or prevention of hypotension, e.g., midodrine. In one embodiment, the method contemplates prevention of hypotension in a subject undergoing hemodialysis. In one embodiment, the method contemplates treatment of hypotension in a subject undergoing hemodialysis.

In one embodiment, the elevated nitric oxide levels are determined by measuring the subject's nitrite/nitrate levels.

In one embodiment, the physiologically compatible compound has the structure of any one of Formulae (I), (I), (Ia), or (IIa).

In another aspect, there is provided a method to determine what treatment is indicated for a subject at risk of developing hypotension associated with hemodialysis. The method includes monitoring the subject's nitric oxide levels, and identifying those subjects which display an elevated level of nitric oxide as suitable for treatment with an effective amount of at least one physiologically compatible compound which, in combination with iron, binds nitric oxide.

In one embodiment, the elevated nitric oxide levels are determined by measuring the subject's nitrite/nitrate levels. In one embodiment, subjects which do not display elevated nitrite/nitrate levels are subjected to standard of care for hypotension associated with hemodialysis. The terms “standard of care” and the like in the context of hypotension associated with hemodialysis refer, as customary in the art, to methods known in the art and described above for the treatment and/or prevention of hypotension associated with hemodialysis, which methods do not include administration of an effective amount of at least one physiologically compatible compound which, in combination with iron, binds nitric oxide.

In one embodiment, the physiologically compatible compound has the structure of any one of Formulae (I), (I), (Ia), or (IIa).

In another aspect, there is provided a method to determine what treatment is indicated for a subject at risk of developing hypotension associated with hemodialysis. The method includes monitoring the subject's endothelial nitric oxide synthase levels, and identifying those subjects which display an elevated level of endothelial nitric oxide synthase as suitable for treatment with an effective amount of at least one physiologically compatible compound which, in combination with iron, binds nitric oxide.

In one embodiment, the physiologically compatible compound has the structure of any one of Formulae (I), (I), (Ia), or (IIa).

In another aspect, there is provided a method to determine whether a subject at risk of developing hypotension associated with hemodialysis is receiving adequate treatment therefor. The method includes administering to those subjects having elevated nitric oxide an effective amount of at least one physiologically compatible compound which, in combination with iron, binds nitric oxide so as to bring the subject's nitric oxide levels within an acceptable range, monitoring the subject's nitric oxide levels, and adjusting the dosage of the physiologically compatible compound which, in combination with iron, binds nitric oxide as needed.

In one embodiment, the physiologically compatible compound has the structure of any one of Formulae (I), (I), (Ia), or (IIa).

In another aspect, there is provided a unit dosage form including a sufficient amount of a physiologically compatible compound which, in combination with iron, binds nitric oxide for administration thereof to a subject in need thereof having hypotension associated with hemodialysis, and a pharmaceutically acceptable carrier therefor. Optionally, the unit dosage form further includes one or more agents known in the art of treatment or prevention of hypotension, e.g., midodrine. The terms “unit dosage form” and the like refer, as customary in the art, to pharmaceutical preparations (e.g., a physiologically compatible compound as disclosed herein) in which the preparation is subdivided into unit doses containing appropriate quantities of the active component(s) (e.g., a physiologically compatible compound described herein, optionally in combination with one or more agents known in the art of treatment or prevention of hypotension, e.g., midodrine). The unit dosage form can be a packaged preparation, the package containing discrete quantities of preparation, such as packeted tablets, capsules, liquids, or powders in vials or ampoules. The unit dosage form can be a capsule, tablet, cachet, vial or ampoule itself, or it can be the appropriate number of any of these in packaged form.

In one embodiment, there is provided a physiologically compatible compound including a compound having the structure of Formula (I) or the structure of Formula (II), as described herein, in a pharmaceutically suitable vehicle (i.e., pharmaceutically acceptable carrier) rendering the compound amenable to oral delivery, transdermal delivery, intravenous delivery, intramuscular delivery, topical delivery, nasal delivery, and the like. The terms “pharmaceutically suitable,” “pharmaceutically acceptable” and the like refer, in the customary sense, to a composition which includes no components at toxic concentrations and would be understood by the clinical practitioner to be generally safe and effective for administration to a subject. Compounds of Formulae (I) or (II) which are free of transition metal cations can be employed directly in the practice of the present invention, or pre-formed dithiocarbamate-transition metal chelates (i.e., compounds of either of Formulae (Ia) or (IIa) having varying ratios of transition metal to dithiocarbamate-species) can be employed in the invention methods.

Depending on the mode of delivery employed, the nitric oxide scavengers contemplated for use herein can be delivered in a variety of pharmaceutically acceptable forms. For example, the scavenger can be delivered in the form of a solid, solution, emulsion, dispersion, micelle, liposome, and the like.

Pharmaceutically acceptable forms of the present invention can be used in the form of a solid, a solution, an emulsion, a dispersion, a micelle, a liposome, and the like, wherein the resulting composition contains one or more of the compounds of the present invention, as an active ingredient, in admixture with an organic or inorganic carrier or excipient suitable for enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. The active compound (e.g., compounds of Formula (I) or Formula (II) as described herein) is included in the pharmaceutical composition in an amount sufficient to produce the desired effect upon the process or condition of disease.

Pharmaceutically acceptable forms containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of a sweetening agent such as sucrose, lactose, or saccharin, flavoring agents such as peppermint, oil of wintergreen or cherry, coloring agents and preserving agents in order to provide pharmaceutically elegant and palatable preparations. Tablets containing the active ingredient in admixture with non-toxic pharmaceutically acceptable excipients may also be manufactured by known methods. The excipients used may be, for example, (1) inert diluents such as calcium carbonate, lactose, calcium phosphate or sodium phosphate; (2) granulating and disintegrating agents such as corn starch, potato starch or alginic acid; (3) binding agents such as gum tragacanth, corn starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,160,452; and 4,265,874, to form osmotic therapeutic tablets for controlled release.

In some cases, formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, for example, peanut oil, liquid paraffin, or olive oil.

The pharmaceutically acceptable forms may be in the form of a sterile injectable suspension. This suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. Buffers, preservatives, antioxidants, and the like can be incorporated as required.

Pharmaceutically acceptable forms contemplated for use in the practice of the present invention may also be administered in the form of suppositories for rectal administration of the drug. These compositions may be prepared by mixing the drug with a suitable non-irritating excipient, such as cocoa butter, synthetic glyceride esters of polyethylene glycols, which are solid at ordinary temperatures, but liquify and/or dissolve in the rectal cavity to release the drug.

Since individual subjects may present a wide variation in severity of symptoms and each drug has its unique therapeutic characteristics, it is up to the practitioner to determine a subject's response to treatment and vary the dosages accordingly.

Typical daily doses, in general, lie within the range of from about 10 μg up to about 100 mg per kg body weight, and, preferably within the range of from 50 μg to 10 mg per kg body weight and can be administered up to four times daily. The daily I.V. dose lies within the range of from about 1 μg to about 100 mg per kg body weight, and, preferably, within the range of from 10 μg to 10 mg per kg body weight.

In another aspect, there is provided kit including a physiologically compatible compound which, in combination with iron, binds nitric oxide, a pharmaceutically acceptable carrier therefor, and instructions for the use thereof in the treatment of a subject in need thereof having hypotension associated with hemodialysis. In one embodiment, the compatible compound is provided in the form of a unit dosage form including a physiologically compatible compound which, in combination with iron, binds nitric oxide, and a pharmaceutically acceptable carrier therefor.

In one embodiment, the physiologically compatible compound has the structure of any one of Formulae (I), (I), (Ia), or (IIa).

### EXAMPLES

The following examples are offered to illustrate, but not to limit the claimed invention.

### Example 1

### NOX-100 as an .NO Scavenger

Compound.

Compound NOX-100 (N-methyl-D-glucamine dithiocarbamate, Sodium MGD) is a sterile-precipitated and dried drug substance having no added buffering, stabilizing, anti-microbial or tonicity agents which can be aseptically reconstituted with Sterile Water for Injection, USP, prior to its intended administration, e.g., intravenous administration. Sodium MGD is a white to slightly yellowish powder with 1.5 molecules of co-crystallized water, and is water soluble up to 35% w/v at approximately 25° C. A 4.5% (w/v) solution of the Sodium MGD in water without any additives affords a near isotonic solution.

Binding.

NOX-100 in the presence of a metal ion (e.g., iron) binds .NO very tightly and prevents this radical species from interacting with other biomolecules. As illustrated in Scheme 1 following, the binding is a two-step process. First, two molecules of NOX-100 combine with Fe²⁺ to form a complex, [(NOX)₂-Fe], which is held together via the planar coordination of the Fe²⁺ by two dithiocarbamates. Next, the [(NOX)₂-Fe] complex tightly binds .NO, forming an .NO-containing complex, [(NOX)₂-Fe—NO]. See e.g., Yokokawa et al, 1995, Id.; Kang et al, 1997, Id.

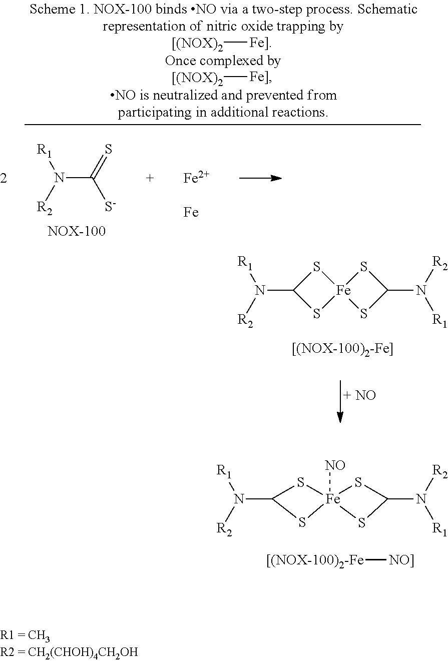

Blood Pressure Studies in Mice.

In mice, intravenous infusion of NOX-100 alone did not affect blood pressure whereas co-administration of NOX-100 with sodium nitroprusside (a .NO donor) reduced the hypotensive effect of nitroprusside. In contrast, other .NO scavengers, such as cross-linked hemoglobins or NOS inhibitors (e.g., N-monomethyl-L-arginine), can cause hypertension in both normal animals and animals with hypotensive shock. The lack of effect on normal blood pressure is advantageous for NOX-100 as a small molecule .NO scavenger.

Pharmacology.

Pharmacological studies have demonstrated the effect of NOX-100 in several animal models. For example, in Wistar rats, shock induced by endotoxin such as LPS (lipopolysaccharide from Salmonella typhosa) is accompanied by severe hypotension, as well known in the art. This hypotension is gradually reversed towards normal blood pressure levels by NOX-100 at a dose of 0.3 mmol/kg. In addition, the survival rate was increased by NOX-100.

Bacterial translocation across damaged intestinal epithelium into the circulation occurs in rats during endotoxin-induced shock, as evidenced by positive bacterial cultures grown from blood, mesenteric lymph nodes, spleen, or liver obtained after LPS challenge. Prior infusion of NOX-100 (200 mg/kg/day) dramatically reduced this bacterial translocation by about 70%.

Moreover, in the rat shock model, liver injury occurs as a result of hypoperfusion and Kupffer cell activation with the release of cytokines by LPS. The three plasma indices of liver injury, ornithine carbamoyltransferase, aspartate transaminase and fibrinogen, were all significantly reduced by NOX-100 treatment (200 mg/kg/day).

Furthermore, the effect of NOX-100 on shock induced by hemorrhage has been demonstrated in a rat model. Treatment of these rats with NOX-100 (0.1 mmol/kg/hour) reduced the blood volume needed to maintain the blood pressure during shock and improved mean arterial pressure after resuscitation. Moreover, this treatment prevented inflammatory gene expression, transcription factor activation, and elevation of serum ornithine carbamoyltransferase (an indicator of hepatic injury) 24 hours after hemorrhagic shock.

Acute/Subchronic Toxicity Studies.

NOX-100 has been evaluated in a number of toxicological studies, including acute (single-dose in rats and dogs; 4-day dosing in rats and dogs), subacute (14-day dosing in rats and dogs) studies, and in vitro mutagenicity testing.

In a preliminary acute single dose study, male Wistar rats were administered NOX-100 intravenously (I.V.) at doses of 30-608 mg/kg or subcutaneous (S.C.) doses of 250-1,000 mg/kg. Each dosing was followed by a 7-day observation period. There was no mortality or overt signs of toxicity noted throughout the 7-day observation period. Another group of male Wistar rats were administered NOX-100 with Fe²⁺ as a single SC dose at 250-1,000 mg/kg. These doses induced no overt toxicities, but dark urine was noted at 60 minutes post-dose in 7 of 8 rats of the 500 mg/kg group and 8 of 8 rats of the 1,000 mg/kg group. Without wishing to be bound by any theory, it is believed that this discoloration was due to the color of the compound at the high dose.

Based on the preliminary I.V. and S.C. studies, doses of 1000, 1450 or 2000 mg/kg of NOX-100 administered over an 8-hour period were tested in albino rats. A total dose of 2000 mg/kg administered over a shorter period of 2 hours was then tested. For all the single dose administrations of NOX-100 infused over 2 or 8 hours, there was no mortality, no treatment-related clinical signs, and no significant effects on body weight.

The high dose of 2000 mg/kg of NOX-100 administered over a period of 2 hours was again tested in a group of rats for 4 consecutive days. The treatment resulted in a slight decrease in red blood cell count, associated with a slight decrease in hemoglobin and hematocrit values. Microscopic examinations revealed that in the group of 5 males dosed at 2000 mg/kg/day over a 2-hour infusion period for 4 consecutive days, cortical tubular basophilia of the kidney, occasionally with tubular dilatation, was observed in 5/5 animals. Mitotic figures in basophilic tubules were also noted. These changes were considered treatment related. At the high dose in this study, the plasma concentrations of NOX-100 determined by HPLC were slightly higher on Day 4 when compared to Day 1, both at 1 and 1.75 hours after infusion. It appeared that female rats had lower plasma concentrations of NOX-100 compared to males.

The safety profile of NOX-100 was further evaluated in Sprague-Dawley rats (10/sex/group for the main study and 24/sex/group for the satellite groups for plasma concentration determination). Rats were administered with NOX-100 at 0, 100, 500 or 2000 mg/kg/day by infusion over a period of 8 hours for 14 days. There were no treatment related deaths in this study. Clinical signs of salivation were noted mostly at 2000 mg/kg/day and occasionally in the lower dose groups. Elevations in blood urea nitrogen and creatinine, increases in urinary output and kidney weights were associated with microscopic changes in the kidney, comprising tubular epithelial basophilia, pyelitis at the papillary tip and hyperplasia of the pelvic transitional epithelium. Microscopic examination also revealed treatment related hyperplasia of the urinary bladder and increased mitotic figures and dilatation of lymphatic vessels in the duodenum at 2000 mg/kg/day. Kidney weights increased in females at 500 mg/kg/day but microscopic changes were only seen in one rat.

The dose of 100 mg/kg/day is considered the no-effect-level in rats. Also, in this study the mean plasma concentrations of NOX-100 remained constant between 4 to 7.75 hours from the start of infusion each day, and the values were similar between Days 1, 7 and 14. There were no differences in the mean steady state plasma concentrations between males and females. A proportional dose concentration relationship was also noted.

When tested in beagle dogs, NOX-100 at 500-2000 mg/kg/day infused over a period of 8 hours for 1 or 4 days generally decreased body weight and food consumption. At the high dose of 2000 mg/kg/day, NOX-100 induced thickening of the duodenal wall and tubular dilatation of the kidney with basophilia in the cortex and vacuolation in the areas of cortical tubules. In this study when plasma concentration of NOX-100 was determined on Days 1 and 4, there were apparent increases in the plasma concentrations obtained at 2 hours from the start of infusion on Day 4 compared to Day 1 both in males and females.

Based on the findings from the 4-day study, doses of NOX-100 at 100, 500 and 1000 mg/kg/day infused over a period of 8 hours for 14 days were evaluated in beagle dogs. One male dog in the high dose (1000 mg/kg/day) group died on Day 6. Histopathological examination of this animal revealed renal lesions consisting of tubular basophilia and dilatation, hyperplasia of the transitional epithelium of the papilla and some vascular hemorrhage or congestion. Therefore the high dose of 1000 mg/kg/day was reduced to 250 mg/kg/day on Days 8-14. Clinical signs of deterioration, reduction in body weight and food intake together with changes in serum chemistry and creatinine clearance, generally at dose levels of 500 or 1000 mg/kg/day, were accompanied by renal lesions seen during histopathological examination. The low dose of 100 mg/kg/day over a period of 8 hours was considered a no-effect level. In this study, the mean plasma concentration of NOX-100 remained constant between 4 to 7.75 hours from the start of infusion on each day and the values were similar throughout Days 1, 7 and 14. There were no differences in the mean steady state plasma concentrations between males and females. A proportional dose concentration relationship was noted.

In preclinical toxicology studies, the drug was administered in doses of up to 2000 mg/kg/day to rats for 14 consecutive days with little observable toxicity. Similarly, in dogs, signs of adverse effects were not observed until doses of 500-2000 mg/kg/day were administered.

The no adverse effect level in dogs (the animal that appeared most sensitive to the effects of NOX-100) was 100 mg/kg given as an 8 hour daily infusion for 14 days. In this study, the NOX-100 was administered as an 11.1 mg/kg bolus loading dose and an 8-hour 11.1 mg/kg/hr infusion maintenance dose. Initial toxicity was seen at 5 times this dose level.

The pharmacokinetics of intravenously administered NOX-100 was evaluated in mice, rats and dogs. Preliminary pharmacokinetic studies were conducted using a bolus injection of 30 mg/kg. Post injection, the drug was rapidly eliminated in all three species in a bi-exponential manner with a beta-t½ of approximately 6, 30 and 40 minutes in the mouse, rat and dog, respectively. The corresponding clearance rates were 26 mL/min·kg in mice, 15±1 mL/min·kg in rats, and 4.1±0.6 mL/min·kg in dogs. The volume of distribution was around 20-30% of body weight and was similar in the three species. This is likely due to the hydrophilic nature of the drug and may reflect the distribution of NOX-100 primarily in the extracellular space.

### Example 2

### Four-Week Repeated Dose Intravenous Toxicity Study of NOX-100 in Rats Followed by a Two-Week Recovery Period

The objective of this study was to investigate the toxicity of NOX-100 when administered as a single intravenous injection once daily for four consecutive weeks to Sprague-Dawley (SPF) rats and to determine the reversibility of changes which were established as treatment related.

NOX-100 was administered intravenously as a daily single dose (“pulse”) (40 mL/kg) at doses of 100, 500, and 2000 mg/kg/day for four weeks in Crj:CD(SD)IGS rats (SPF). The Control group was treated with saline (n=60 rats per gender−balanced randomization across all groups.) Following termination of treatment, an additional group of rats in the Control and highest dose group were held for a period of two weeks in order determine if the potential treatment related changes were reversible.

In the 2000 mg/kg group, a decrease in locomotor activity, dyspnea, and lateral position were observed immediately after dosing on Day 15 in one male animal. These findings disappeared within 30 minutes after dosing and were not observed thereafter. These changes were not observed in any other animals. Salivation was observed in the 500 mg/kg group and the 2000 mg/kg group. Irregular respiration was observed in all animals, including the Control group animals with the frequency of occurrence higher in males and females of the 2000 mg/kg group. There were no significant differences between the Control and Treatment groups in food consumption or body weight gain. Animals in the 2000 mg/kg group exhibited increased water consumption as compared to the Control group. This was measured from Day 15 of the study until the conclusion of the treatment period.

No differences were observed during the recovery period. Hematological changes included decreased erythrocyte count and increased reticulocyte count in the 2000 mg/kg group, a decreased hematocrit in the females in the 500 and 2000 mg/kg group and a decrease of hemoglobin in the females in the 2000 mg/kg group. Blood chemistry observations included decreased iron observed in the 2000 mg/kg group, an increased sodium and chloride and increased albumin in the males of the 2000 mg/kg group and an increased cholesterol level in the females of the 2000 mg/kg group. A decrease in the urine chloride and sodium was observed in the 2000 mg/kg group. In addition, a tendency toward increased urine volume and decreased specific gravity was seen in the 2000 mg/kg group.

No changes were observed in the ophthalmological examinations. Organ weight observations included decreased absolute thymus weights observed in the males, increased absolute kidney weight in the females and increased relative liver and kidney weights in the males and females of the 2000 mg/kg group. Histopathological examinations of the kidney revealed renal papillary edema and an increased number of animals with basophilic proximal tubules in the 2000 mg/kg group. At the end of the recovery period, renal papillary edema and basophilic proximal tubules were observed in the 2000 mg/kg group. Findings in the urinary bladder consisted of diffuse hyperplasia of the mucosal epithelium in the females of the 500 and 2000 mg/kg groups. At the end of the recovery period, no clear-cut reversibility of this change was evident.

The treatment-related toxic changes in the 2000 mg/kg group included salivation, increased kidney weight, renal papillary edema, increased number of animals with basophilia of the proximal tubules in the kidney, diffuse hyperplasia of the mucosal epithelium of the urinary bladder, decreased serum iron, increased serum cholesterol, chloride and sodium, decreased erythrocyte count, hematocrit and hemoglobin, and turbid urine. Findings in the 500 mg/kg group included salivation, diffuse hyperplasia of the mucosal epithelium of the urinary bladder, and decreased hematocrit.

Among the changes observed in this study, reversibility after a two week period could not be confirmed for diffuse hyperplasia of the mucosal epithelium of the urinary bladder, and increased number of animals with basophilia of the proximal tubules in the kidney, and renal papillary edema.

There were no findings in either sex at the 100 mg/kg dose, and the no adverse effect level was established at 100 mg/kg.

### Example 3

### Four-Week Repeated Dose Intravenous Toxicity Study of NOX-100 in Beagle Dogs Followed by a Two-Week Recovery Period

The objective of this study was to investigate the toxicity of NOX-100 when administered as a single intravenous injection once daily for four consecutive weeks to beagle dogs and to determine the reversibility of changes which were established as treatment related. In addition, the systemic exposure of the animals to the test substance was determined and evaluated for correlation with dose and time after starting treatment.

NOX-100 was administered intravenously as a daily single dose (20 mL/kg administered over approximately 2.5 minutes) of 100, 300, and 1000 mg/kg for four weeks in Beagle dogs (n=17 dogs per gender−unbalanced randomization across treated groups). Following the termination of treatment, an additional group of dogs in the Control and highest dose group were held for a period of two weeks in order to determine if the potential treatment related changes were reversible.

One male dog in the 1000 mg/kg dose group became moribund six days after starting treatment. In the moribund animal, vomiting occurred on Days 1 to 6, no stools were passed on Days 4 to 6, decreased locomotor activity was noted on Days 5 to 6 and hypothermia and lateral position was noted on Day 6. Necropsy revealed an increased absolute and relative weight of the liver, kidneys and adrenals with a decrease noted in the thymus weight. Histopathological examination revealed moderate basophilia of the proximal renal tubules, pelvic mucosal epithelial hyperplasia, hyaline droplets in the proximal tubular epithelium and pelvic mucosal epithelium in the kidneys. The renal papilla was for the most part necrotic. Other findings included follicular atrophy of the mesenteric lymph node, acute atrophy of the thymus, glycogen accumulation in the liver, erosion of mucosa in the urinary bladder, and hypertrophy of cortical cells in the fascicular zone of the adrenals. Laboratory results on the day of necropsy included, an increased white cell count with increased segmented neutrophils and an increase in gamma GT, ALP, urea nitrogen, creatinine, glucose, cholesterol, phospholipids, triglyceride, total protein, albumin, inorganic phosphorus and magnesium and a decrease in sodium and chloride.

Clinical observations for the animals completing the study revealed vomiting in the 100 mg/kg or higher dose groups and salivation in the 1000 mg/kg group. The incidence of vomiting was higher after dosing in the Treated groups than in the group which was not dosed. There were no changes noted in body weight, food consumption, hematology, EKG, ophthalmology or organ weights.

Laboratory determinations of sodium were increased on both sexes of the 1000 mg/kg group. Urinalysis observations included a higher incidence of ketones in the 300 and 1000 mg/kg groups. A decrease in specific gravity and increase in urine volume was noted in the all Treated groups. Decreased urine chloride values were noted in the 1000 mg/kg group.

Necropsy showed enlargement in the liver in one male of the 1000 mg/kg group and enlargement of lymph follicles in the urinary bladder in one female each of the 1000 and 300 mg/kg groups. There were no macroscopic changes due to study substance in the 100 mg/kg group.

Histopathological examination revealed basophilic proximal tubules in the kidneys in the 1000 mg/kg group with the observation of necrotic epithelial cells in the effected tubules of two animals. Hyperplasia of the pelvic transitional epithelium of the renal papilla was also noted in these two animals. Inflammatory cell infiltration in the pelvic mucosa, and erosion of mucosa in the urinary bladder was observed in the animals of both sexes at the 1000 mg/kg group. Inflammatory cell infiltration and hyperplasia of mucosal epithelium were observed in the submucosal tissue of the urinary bladder in one female each of the 300 and 1000 mg/kg groups.

There were no microscopic changes due to study substance in the 100 mg/kg group.

Histopathological changes in the kidneys and urinary bladder were nearly reversed and other changes were reversed completely or showed a tendency for reversal after a two week cessation of study treatment.

Systemic exposure to NOX-100 showed a linear dose dependent increase in blood levels over the period of treatment with no evidence of accumulation after tour weeks of repeat dosing. Additionally, there was no gender related difference in systemic exposure.

The results of this study suggest that the main toxicological findings for NOX-100 are in the kidneys and urinary bladder with apparent adaptive changes in the liver. Under the conditions of this study, the no adverse effect level is less than 100 mg/kg due to the observations of transient vomiting, urine specific gravity decrease, and increased urine volume observed in the lowest (100 mg/kg) dose group. It will be noted that the dosing in this trial was done by “pulse” IV injection as opposed to the eight hour infusion used in the 14 day trial described above.

### Example 4

### Human Clinical Studies

A prior study of NOX-100 carried out over 7 dose escalations with 4 subjects per group did not reveal any clinically significant toxicity at any dose. In that study, the drug was given to subjects who had at least 3 hypotensive episodes during hemodialysis in the prior month. Subjects received the drug during hemodialysis when systolic blood pressure (SBP) had fallen at least 15 mmHg. The following trends appeared between drug and placebo periods. Firstly, there were no apparent differences in heart rate, diastolic blood pressure, number of interventions for low blood pressure or number of symptoms. And secondly, at the three highest doses administered, there was a trend for SBP to be higher after drug than after placebo during the period from 30 to 120 minutes following drug administration. The SBP over this time period averaged 4 mmHg higher for drug than for placebo with a standard deviation of 16 mmHg. Thus, this study showed that blood pressure following an initial hypotensive episode is quite variable. As hypotension worsened, the patients developed several different symptoms that were not predictable for any given patient. Clinical interventions intended to relieve hypotension were also variable and were applied at inconsistent times from patient to patient. This was a result of long standing practices established by patient and nurse to avoid severe symptoms. For example, patients would sometimes request an intervention that they thought would be helpful and nurses would sometimes institute an intervention when they thought that a blood pressure trend might soon lead to symptoms. It was felt unethical to intervene in this process, since the aim was to avoid patient discomfort.

Nonetheless, the NOX-100 study referred to above provides guidance for future studies. The symptoms patients expressed were so variable that it is impractical to classify patients according to the presence of any specific symptom. For example, symptoms might be classified as being present or absent. Thus, it was deemed impractical to try to regulate the type of intervention given to treat hypotension. A similar fall in blood pressure might be treated with a slow and weak intervention, such as turning off hemodialysis ultrafiltration, or might be treated with administration of intravenous saline. Interventions might be classified as none or administered, however.

Pharmacokinetics (PK).

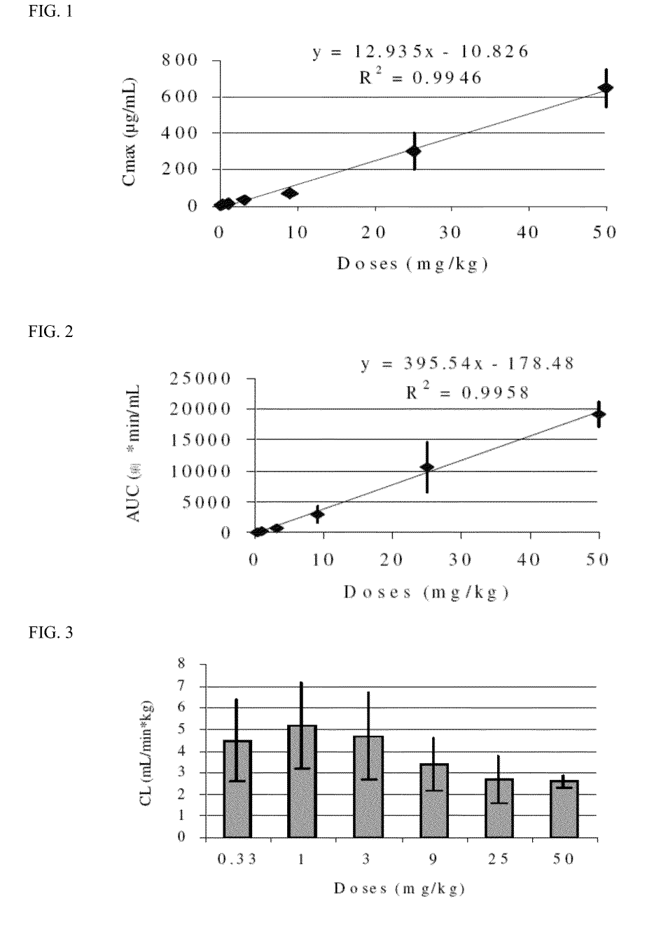

As depicted in FIGS. 1 and 2, the plasma levels of NOX-100 as represented by Cₘₐₓ and Area Under the Curve (AUC), as well known in the art, appeared to be proportional to the dose. It also appeared that the plasma clearance of NOX-100 during dialysis decreased slightly with higher doses but the difference was not statistically significant. See FIG. 3. Using the averaged clearance from the 1, 9, and 50 mg/kg cohorts, the plasma clearance during dialysis was found to be approximately 3.3 mL/min·kg.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for the prevention and/or treatment of hypotension associated with hemodialysis, said method comprising administering to a subject in need thereof an effective amount of at least one physiologically compatible compound which, in combination with iron, binds nitric oxide.

2. The method of claim 1 wherein said subject in need thereof has elevated nitric oxide levels.

3. The method of claim 2 wherein said elevated nitric oxide levels are determined by measuring the subject's nitrite/nitrate levels.

4. The method of claim 3 wherein said subject in need thereof has nitrite/nitrate levels greater than 50 micromolar.

5. The method of claim 1 wherein said physiologically compatible compound which, in combination with iron, binds nitric oxide is a nitric oxide scavenger.

6. The method of claim 5 wherein said nitric oxide scavenger is a dithiocarbamate-containing nitric oxide scavenger.

7. The method of claim 6 wherein said dithiocarbamate-containing nitric oxide scavenger has the structure of Formula (I) or (II) as follows:
[R¹R²N—C(S)—S⁻]ₓM^{+1, +2, +3} (I)
wherein:
each of R¹ and R² is independently selected from a C₁ up to C₁₈ alkyl, substituted alkyl, cycloalkyl, substituted cycloalkyl, heterocyclic, substituted heterocyclic, alkenyl, substituted alkenyl, alkynyl, substituted alkynyl, aryl, substituted aryl, heteroaryl, substituted heteroaryl, alkylaryl, substituted alkylaryl, arylalkyl, substituted arylalkyl, or R¹ and R² can cooperate to form a 5-, 6- or 7-membered ring including N, R¹ and R²;
x is 1, 2 or 3; and
M is a monovalent cation when x is 1, or M is a physiologically compatible divalent or trivalent transition metal cation when x is 2 or 3; or
M^{+1, +2, +3}[⁻S—C(S)—NR¹]ₓ—R³—[R¹N—C(S)—S⁻]ₓM^{+1, +2, +3} (II).
wherein:
each R¹ is independently selected from a C₁ up to C₁₈ alkyl, substituted alkyl, cycloalkyl, substituted cycloalkyl, heterocyclic, substituted heterocyclic, alkenyl, substituted alkenyl, alkynyl, substituted alkynyl, aryl, substituted aryl, heteroaryl, substituted heteroaryl, alkylaryl, substituted alkylaryl, arylalkyl, or substituted arylalkyl;
R³ is a divalent moiety selected from alkylene, substituted alkylene, oxyalkylene, substituted oxyalkylene, alkenylene, substituted alkenylene, arylene, substituted arylene, alkylarylene, substituted alkylarylene, aralkylene, or substituted aralkylene;
x is 1, 2 or 3; and
M is a monovalent cation when x is 1, or M is a physiologically compatible divalent or trivalent transition metal cation when x is 2 or 3.

8. The method of claim 7 wherein:
said dithiocarbamate-containing nitric oxide scavenger has the structure of Formula (I);
each of R¹ and R² is independently selected from a C₁ up to C₆ alkyl or substituted alkyl;
x is 1; and
M is a monovalent cation selected from NH₄⁺, Na⁺ or K⁺.

9. The method of claim 7 wherein:
said dithiocarbamate-containing nitric oxide scavenger has the structure of Formula (I);
each of R¹ and R² is independently selected from a C₁ up to C₆ alkyl or substituted alkyl;
x is 2 or 3; and
M is selected from Fe⁺² or Fe⁺³.

10. The method of claim 7 wherein said dithiocarbamate-containing nitric oxide scavenger is N-methyl-D-glucamine dithiocarbamate (NOX-100).

11. The method of claim 1 wherein said at least one physiologically compatible compound which, in combination with iron, binds nitric oxide is administered to said subject prior to the manifestation of symptoms of hypotension.

12. A method for identifying hemodialysis subjects for whom treatment with at least one physiologically compatible compound which, in combination with iron, binds nitric oxide is indicated, said method comprising:
monitoring the subject's nitric oxide levels, and
selecting those subjects having elevated nitric oxide levels for treatment with an effective amount of at least one physiologically compatible compound which, in combination with iron, binds nitric oxide sufficient to maintain the subject's nitric oxide levels within an acceptable range.

13. The method of claim 12 wherein said elevated nitric oxide levels are determined by measuring the subject's nitrite/nitrate levels.

14. The method of claim 1 wherein said at least one physiologically compatible compound which, in combination with iron, binds nitric oxide is administered to said subject prior to the manifestation of symptoms of hypotension.

15. A method to determine what treatment is indicated for a subject at risk of developing hypotension associated with hemodialysis, said method comprising:
monitoring the subject's nitric oxide levels, and
identifying those subjects which display an elevated level of nitric oxide as suitable for treatment with an effective amount of at least one physiologically compatible compound which, in combination with iron, binds nitric oxide.

16. The method of claim 15 wherein said elevated nitric oxide levels are determined by measuring the subject's nitrite/nitrate levels.

17. The method of claim 15 wherein those subjects which do not display elevated nitrite/nitrate levels are subjected to standard of care for hypotension associated with hemodialysis.

18. A method to determine what treatment is indicated for a subject at risk of developing hypotension associated with hemodialysis, said method comprising:
monitoring the subject's endothelial nitric oxide synthase levels, and
identifying those subjects which display an elevated level of endothelial nitric oxide synthase as suitable for treatment with an effective amount of at least one physiologically compatible compound which, in combination with iron, binds nitric oxide.

19. A composition comprising:
a physiologically compatible compound which, in combination with iron, binds nitric oxide, and
midodrine.

20. A method for the prevention and/or treatment of hypotension associated with hemodialysis, said method comprising administering to a subject in need thereof an effective amount of a composition according to claim 19.

21. A method for the prevention and/or treatment of hypotension associated with hemodialysis, said method comprising administering to a subject in need thereof:
an effective amount of at least one physiologically compatible compound which, in combination with iron, binds nitric oxide, and
midodrine,
wherein said compound and midodrine are administered separately or together.
